(19) United States
(12) Patent Application Publication
(10) Pub. No.: US 2004/0197416 A1
(43) Pub. Date: Oct. 7, 2004

(54) Nanocapsules containing a steroid for cosmetic compositions

(76) Inventors: Jean-Thierry Simonnet, Paris (FR); Pascal Richart, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/484,429

(22) PCT Filed: Jul. 18, 2002
(86) PCT No.: PCT/FR02/02572

(30) Foreign Application Priority Data
Jul. 27, 2001 (FR) 01/10114

(51) Int. Cl. A61K 9/16; A61K 9/50

(52) U.S. Cl. 424/490

(57) ABSTRACT

The present invention relates to an aqueous suspension of nanocapsules containing, in an aqueous medium, nanocapsules comprising a polymer shell and a lipid core containing an oily solvent, characterized in that the said lipid core contains at least one steroid chosen from: DHEA, its chemical and biological precursors and its chemical and metabolic derivatives, and in that the said oily solvent comprises at least one compound chosen from certain classes of solvents for DHEA.

The invention also relates to a cosmetic and/or dermatological composition comprising the said suspension of nanocapsules in a physiologically acceptable medium.

Finally, the invention relates to the cosmetic and dermatological uses of this composition, especially for preventing or treating the signs of ageing of the skin.

The present invention relates to an aqueous suspension of nanocapsules containing at least one steroid chosen from DHEA and its derivatives and precursors, and also to cosmetic and/or dermatological compositions containing them and to the cosmetic and dermatological uses of these compositions.

DHEA, or dehydroepiandrosterone, is a natural steroid produced essentially by the adrenal glands. Exogenous DHEA, administered topically or orally, is known for its capacity to promote keratinization of the epidermis (JP-07 196 467) and to treat dry skin by increasing the endogenous production and the secretion of sebum, thus reinforcing the skin's barrier effect (U.S. Pat. No. 4,496,556). The use of DHEA for overcoming dermal atrophy by inhibiting the loss of collagen and of connective tissue has also been described in patent U.S. Pat. No. 5,843,932. Finally, the Applicant has demonstrated the capacity of DHEA to combat the weathered appearance of the skin (FR 00/00349), to modulate the pigmentation of the skin and the hair (FR 99/12773) and to combat atrophy of the epidermis (FR 00/06154). These properties of DHEA make it a candidate of choice as an anti-ageing active agent.

Among the DHEA metabolites, particular attention has been paid in recent years to 7α-hydroxy DHEA. Specifically, it has been demonstrated that this metabolite, which does not have the hormonal activity of DHEA, makes it possible to increase the proliferation of fibroblasts and the viability of human keratinocytes, and has free-radical-scavenging effects (WO 98/40074). It has also been demonstrated on rats (WO 00/28996) that 7α-hydroxy DHEA increases the thickness of the dermis and the elastin and collagen content of the skin. It has thus been suggested to use this DHEA metabolite to prevent and/or treat the harmful effects of UV on the skin, to combat wrinkles and to increase the firmness and tonicity of the skin.

In the same way as many other cosmetic active agents, DHEA and its derivatives and precursors have the drawback of being very sparingly soluble in the solvents conventionally used in cosmetics. Now, the use of these active agents in dissolved form in cosmetic and/or dermatological supports is desirable since it leads to better bioavailability in the skin than crystallized forms whose crystal size is poorly controlled.

For the purposes of the present patent application, the term “bioavailability” means the molecular penetration of the active agent concerned into the live layers of the skin and in particular of the epidermis. It will be sought to ensure that the penetrated concentration is as high as possible, so as to increase the amount of active agent arriving as far as the live layers of the skin.

The term “dissolved form” means a dispersion of the steroid in a liquid, in free molecular form, in particular in non-complexed form. No crystallization of the steroid should be visible to the naked eye or by cross-polarization optical microscopy.

It is possible to dissolve DHEA and its derivatives and precursors in certain solvents, but, to do this, it is necessary to have very high concentrations of solvents in order to dissolve large amounts of DHEA and its derivatives and precursors. However, it is preferable to minimize, in cosmetic or dermatological compositions, the amount of these “good solvents”, which may show a poor level of harmlessness or may be in relatively unpleasant forms (sticky, greasy, etc. forms).

For this reason, most of the products currently available have contents of DHEA and/or derivatives that are less than 0.4% and usually less than 0.1% by weight, the DHEA also being in crystallized form. There is thus still a need for products based on DHEA or analogues in dissolved form at relatively high concentration, in a cosmetically acceptable support.

One advantageous approach for solving this problem of insolubility of certain cosmetic active agents consists in forming, with these molecules of generally lipophilic active agents, particles of very small size (less than a micron), which are known as nanospheres. These are rigid, solid particles consisting either of the active agent alone or of a combination of the active agent with one or more polymers. They are less than one micron in size. This solution has especially been applied to DHEA in patent application FR 00/15686 in the name of the Applicant.

The encapsulation or absorption of lipophilic active principles in particles of submicron size has been known for several years and is widely used in particular in cosmetology and dermatology, since these particles, known as nanoparticles, are capable of crossing the superficial layers of the stratum corneum and of penetrating into the upper layers of the live epidermis to release the active principle therein. This penetration into deeper layers broadens the space of action of the active principles and protects them from rapid removal by simple rubbing.

The term “nanoparticles” primarily encompasses two different systems: “nanospheres” consisting of a polymer matrix in which the active principle is absorbed and/or adsorbed and/or mixed, and “nanocapsules” with a structure of core-shell type, i.e. a structure consisting of a lipid core that is liquid at room temperature, which is formed or contains the active principle in pure or dissolved form, this core being encapsulated in a continuous protective shell that is insoluble in the medium.

To the Applicant's knowledge, it has never been suggested to improve not only the bioavailability but also the solubility of DHEA and its analogues by introducing nanocapsules into the lipophilic core.

Now, the Applicant has discovered that it is possible to obtain such nanocapsules containing at least one steroid chosen from DHEA and/or its analogues dissolved in solvents in amounts higher than the existing amounts, and even going beyond the usual limiting recrystallization level. The nanocapsules thus obtained make it possible to provide aqueous suspensions of DHEA and/or derivatives or precursors, without recrystallization, which it is then possible to introduce into the cosmetic supports conventionally used.

One subject of the present invention is thus an aqueous suspension of nanocapsules containing, in an aqueous medium, nanocapsules comprising a polymer shell and a lipid core containing an oily solvent, characterized in that the said lipid core contains at least one steroid chosen from: DHEA, its chemical and biological precursors and its chemical and metabolic derivatives, and in that the said oily solvent comprises at least one compound chosen from:

2-alkyl alkanols containing from 12 to 36 carbon atoms, or an ester of such an alcohol,

fatty acid esters in which the acid function contains from 8 to 26 carbon atoms and the alcohol function contains from 2 to 8 carbon atoms,

fatty alkyl esters in which the acid function contains from 2 to 8 carbon atoms and the alcohol function contains from 8 to 26 carbon atoms,

esters of N-acyl amino acids and of fatty alcohols,

triglycerides formed from at least one acid containing from 6 to 20 carbon atoms, and/or plant oils containing them,

liquid ethers of fatty alcohols and of polypropylene glycol, and

tocopherol and/or tocopheryl esters.

DHEA has the formula (I) below:

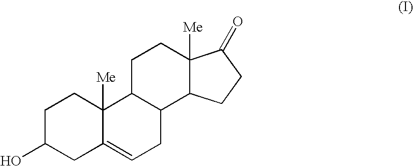

It is available, for example, from the company Akzo Nobel.

The expression “DHEA precursors” means its biological precursors that may be converted into DHEA during metabolism, and also its chemical precursors that may be converted into DHEA by exogenous chemical reaction. Examples of biological precursors are Δ5-pregnenolone, 17α-hydroxypregnenolone and 17β-hydroxypregnenolone sulphate, this list not intended to be limiting. Examples of chemical precursors are sapogenins and derivatives thereof such as diosgenin (or 5-spirostene-3β-ol), hecogenin, hecogenin acetate, smilagenin and sarsapogenin, and also natural extracts containing them, in particular fenugreek and extracts of Dioscorea plants such as extract of wild yam root, this list not intended to be limiting.

The expression “DHEA derivatives” means both its metabolic derivatives and its chemical derivatives. Metabolic derivatives that may especially be mentioned include Δ5-androstene-3,17-diol and Δ4-androstene-3,17-dione, and also 7α-OH DHEA, 7β-OH DHEA and 7-keto-DHEA, this list not intended to be limiting. 7α-OH DHEA is preferred for use in the present invention. A process for preparing this compound is described in particular in patent applications FR 2 771 105 and WO 94/08588.

Chemical derivatives that may also be mentioned include DHEA salts and in particular water-soluble salts such as DHEA sulphate. Mention may also be made of esters such as hydroxycarboxylic acid esters of DHEA, in particular those described in U.S. Pat. No. 5,736,537 or other esters such as DHEA salicylate, DHEA acetate, DHEA valerate (or n-heptanoate) and DHEA enanthate. Mention may also be made of DHEA derivatives (DHEA carbamates, DHEA 2-hydroxymalonate esters and DHEA amino acid esters) described in patent application FR 00/03846 in the name of the Applicant. 3-Alkyl esters of 7-oxo-DHEA, in particular 3β-acetoxy-7-oxo-DHEA, may also be mentioned.

Other chemical DHEA derivatives that are suitable for use in the present invention are the derivatives of formula (1):

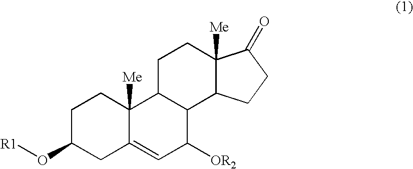

in which:

R₁ and R₂ are chosen independently from:

a saturated or unsaturated, linear, branched or cyclic C₁-C₁₂ alkyl group optionally containing one or more hetero atoms, and optionally substituted with one or more groups chosen from —OR′ and/or —SR′ and/or —COOR′ and/or —NR′R′ and/or halogen and/or sulphate and/or phosphate and/or aryl and/or heterocycle, the said heterocycle advantageously being chosen from an indole, a pyrimidine, a piperidine, a morpholine, a pyran, a furan, a piperazine and a pyridine;

an alkylcarbonyl group, the C₁-C₂₄ alkyl portion of which is saturated or unsaturated, linear, branched or cyclic, and optionally substituted with one or more groups chosen from —OR′ and/or —SR′ and/or —COOR′ and/or —NR′R′ and/or halogen and/or sulphate and/or phosphate and/or aryl and/or heterocycle, the said heterocycle advantageously being chosen from an indole, a pyrimidine, a piperidine, a morpholine, a pyran, a furan, a piperazine and a pyridine;

an arylcarbonyl group, preferably a phenylcarbonyl group, or an arylalkylcarbonyl group, preferably a benzylcarbonyl group, optionally substituted with one or more groups —OR′ and/or —SR′ and/or —COOR′ and/or —NR′R′ and/or halogen and/or aryl and/or heterocycle;

a group O═P(OH)OR′;

a group (O)₂SOR′;

a trialkylsilyl group (SiR′₃) in which the 3 groups R′ may be identical or different;

a carbonyloxyalkyl group (R′OCO);

a carbonylaminoalkyl group (R′NHCO);

in which R′ is chosen from a hydrogen atom, a saturated or unsaturated, linear, branched or cyclic C₁-C₁₂ and preferably C₁-C₆ alkyl group optionally containing one or more hetero atoms, optionally functionalized with one or more groups —OR″, —COOR″, halogen, —NR″R″; or

with an aryl group, preferably a phenyl group, optionally functionalized with one or more groups —OR″, —COOR″, halogen or —NR″R″;

R″ representing a hydrogen atom or a saturated or unsaturated, linear, branched or cyclic alkyl chain, preferably of C₁-C₆,

it being understood that, in each of the groups —NR′R′ and —NR″R″, the substituents R′ or R″, respectively, are identical or different.

Among the derivatives of formula (1) that may be mentioned in particular are 7-OH-DHEA diesters and more preferably 3-O-acetyl-7-benzoyloxydehydroepiandro-sterone, which is commercially available from the company Gattefosse under the trade name 3-acetoxy-7-benzoate DHEA.

According to one advantageous aspect of the invention, the steroid content in the nanocapsules according to the invention is higher than the maximum solubility level of the steroid in the oily solvent.

The nanocapsules according to the present invention are generally of small size in order to obtain optimum bioavailability of the steroid. Preferably, these nanocapsules are between 10 nm and 1000 nm and more particularly between 30 nm and 500 nm in size.

Various types of nanocapsules may be used according to the present invention. Examples that may be mentioned include the nanocapsules described in patent application EP-0 274 961, the nanocapsules provided with a lamellar coating described in patent application EP-0 780 115, nanocapsules whose water-insoluble continuous polymer shell consists of polyesters, as described in patent applications EP-1 025 901, FR-2 787 730 and EP-1 034 839, or alternatively the biodegradable nanocapsules described in patent application FR-2 659 554, or the non-biodegradable nanocapsules described in patent application WO-93/05753.

Nanocapsules made of biodegradable polymers penetrate into the skin and degrade in the epidermis under the action of the enzymes present therein, whereas nanocapsules made of non-biodegradable polymers penetrate only into the superficial layers of the stratum corneum and are naturally eliminated during the renewal of the skin.

The use of any such type of polymer thus depends on the mode of action intended for the steroid, and thus on the desired cosmetic or dermatological effect.

1) Biodegradable polymers that may be used include any polymer capable of being degraded by the enzymes of the skin, and especially those described in document EP-A-447 318. Biodegradable polymers that may be mentioned in particular include poly-L- and DL-lactides and polycaprolactones, polyglycolides and copolymers thereof, and also polymers derived from the polymerization of alkyl cyanoacrylate (the alkyl chain containing from 2 to 6 carbon atoms).

2) Among the other biodegradable polymers that may be used to form the nanocapsules according to the invention, mention may be made of synthetic water-dispersible anionic polymers such as in particular polyesters, poly(esteramides), polyurethanes and vinyl copolymers, all bearing carboxylic acid and/or sulphonic acid functions, and natural water-dispersible anionic polymers chosen from shellac resin, sandarac gum and dammar resins.

The anionic polyesters are obtained by polycondensation of aliphatic, cycloaliphatic and/or aromatic dicarboxylic acids and aliphatic, cyclo-aliphatic and/or aromatic diols or polyols, a certain number of these diacids and diols also bearing a carboxylic acid or sulphonic acid function in free form or in the form of a salt.

Dicarboxylic acids which may be mentioned are succinic acid, glutaric acid, adipic acid, pimelic acid, suberic acid, sebacic acid, terephthalic acid, isophthalic acid or the anhydride thereof. Aliphatic diols which may be mentioned are ethylene glycol, diethylene glycol, triethylene glycol and tetraethylene glycol, di(hydroxymethyl)cyclohexane, dimethylolpropane and 4,4′-(1-methylpropylidene)bisphenol.

The polyol monomers are, for example, glycerol, pentaerythritol or sorbitol. The comonomers which allow anionic groups to be introduced are, for example, dimethylolpropionic acid, trimellitic acid or mellitic anhydride, or a diol or dicarboxylic acid compound also bearing a group SO₃M in which M represents a hydrogen atom or an alkali metal ion, such as sodium 1,5-dihydroxypentane-3-sulphonate or sodium 1,3-dicarboxybenzene-5-sulphonate.

The poly(ester amides) which can be used as other synthetic water-dispersible anionic polymers have a structure similar to that of the polyesters described above, but also contain units derived from a diamine such as hexamethylenediamine, meta- or paraphenylenediamine, or from an amino alcohol such as methanolamine.

According to one preferred embodiment of the invention, the water-dispersible anionic polymer is chosen from aromatic, cycloaliphatic and/or aliphatic polyesters bearing sulphonic acid functions, i.e. copolyesters comprising at least a number of units derived from isophthalic acid, from sulphoaryldicarboxylic acid and from diethylene glycol. Among these, mention may be made most particularly of polyesters comprising units derived from isophthalic acid, from sulphoisophthalic acid, from diethylene glycol and from 1,4-di(hydroxymethyl)cyclohexane, such as those sold under the names AQ29, AQ38, AQ48 Ultra, AQ55S, AQ1350, AQ1045, AQ1950 and AQ14000 by the company Eastman Chemical.

These polyesters can also contain units derived from ethylene glycol, from triethylene glycol and/or from tetraethylene glycol and from terephthalic acid, such as those sold under the names Polycare PS 20, Polycare PS 30 and Polycare PS 32 by the company Rhône-Poulenc. The proportion of units derived from sulphoisophthalic acid is generally between 2 and 20% by weight.

3) According to one variant of the invention, the biodegradable polymer forming the shell of the nanocapsules may be a polyester of the poly(alkylene adipate) type, i.e. a homopolymer of adipic acid and of an alkanediol, or a copolymer of linear or branched poly(ester ether) type, obtained from adipic acid and from one or more alkanediols and/or etherdiols and/or triols. The alkanediols used for the preparation of the said poly(alkylene adipates) are C₂₋₆ alkanediols with a linear or branched chain, chosen from ethylene glycol, propylene glycol, 1,3-propanediol, 1,4-butanediol, 1,5-pentanediol, 1,6-hexanediol and neopentyl glycol. The etherdiols are di-, tri- or tetra(C₂₋₄ alkylene) glycols such as diethylene glycol, triethylene glycol, tetraethylene glycol, dipropylene glycol, tripropylene glycol, tetrapropylene glycol or dibutylene glycol, tributylene glycol or tetrabutylene glycol. The triols used are generally chosen from glycerol, trimethylolethane and trimethylolpropane.

The fraction of the branching units derived from the above triols generally does not exceed 5 mol % relative to the total amount of units derived from diols and triols. According to one preferred embodiment of the present invention, the shell of the nanocapsules is formed by a poly(ethylene adipate) or a poly(butylene adipate).

The poly(alkylene adipates) used in the present invention have a weight-average molar mass (measured by gel permeation chromatography) preferably of between 2 000 and 50 000 and more preferably between 5 000 and 15 000.

A whole range of products of various chemical compositions and of various molar masses is sold under the name Fomrez® by the company Witco. The company Scientific Polymer Products sells under the name Poly(ethylene) Adipate® a poly(ethylene adipate) with a weight-average molar mass (determined by GPC) of about 10 000.

4) Another class of polymers that may be used to form the shell of the nanocapsules according to the invention consists of dendritic polymers. These are hyperbranched polymers with the chemical structure of a polyester and which are terminated with hydroxyl groups optionally modified with at least one chain-terminating agent. The structure and preparation of such polymers is described in patent applications WO-A-93/17060 and WO 96/12754.

More specifically, the dendritic polymers used in the compositions of the present invention can be defined as being highly branched macromolecules of polyester type, consisting

of a central unit derived from an initiator compound bearing one or more hydroxyl functions (a),

of chain-extending units derived from a chain-extending molecule bearing a carboxyl function (b) and at least two hydroxyl functions (c), each of the hydroxyl functions (a) of the central molecule being the starting point of a polycondensation reaction (by esterification) which starts with the reaction of the hydroxyl functions (a) of the central molecule with the carboxyl functions (b) of the chain-extending molecules, and then continues by reaction of the carboxyl functions (b) with the hydroxyl functions (c) of the chain-extending molecules.

A “generation X” dendrimer refers to a hyperbranched polymer prepared by X condensation cycles, each cycle consisting in reacting all of the reactive functions of the central unit or of the polymer with one equivalent of a chain-extending molecule. The initiator compound bearing one or more hydroxyl functions and forming the central unit around which the dendritic structure will be constructed is a monohydroxy, dihydroxy or polyhydroxy compound. It is generally chosen from

(a) a monofunctional alcohol,

(b) an aliphatic, cycloaliphatic or aromatic diol,

(c) a triol,

(d) a tetrol,

(e) a sugar alcohol,

(f) anhydro-ennea-heptitol or dipentaerythritol,

(g) an α-alkylglycoside,

(h) a polyalkoxylated polymer obtained by polyalkoxylation of one of the alcohols (a) to (g), with a molar mass of not more than 8000.

As examples of preferred initiator compounds for preparing the dendritic polymers used in the present invention, mention may be made of ditrimethylolpropane, ditrimethylolethane, dipentaerythritol, pentaerythritol, an alkoxylated pentaerythritol, trimethylolethane, trimethylolpropane, an alkoxylated trimethylolpropane, glycerol, neopentyl glycol, dimethylolpropane or 1,3-dioxane-5,5-dimethanol.

These hydroxylated initiator compounds forming the central unit of the future dendrimer are reacted with molecules referred to as chain-extending molecules, which are compounds of monoacidic diol type chosen from:

monocarboxylic acids comprising at least two hydroxyl functions, and

monocarboxylic acids comprising at least two hydroxyl functions, one or more of which bear(s) a hydroxyalkyl substituent.

Preferred examples of such compounds are dimethylolpropionic acid, α,α-bis(hydroxymethyl)butyric acid, α,α,α-tris(hydroxymethyl)acetic acid, α,α-bis(hydroxymethyl)valeric acid, α,α-bis(hydroxy)-propionic acid and 3,5-dihydroxybenzoic acid.

According to one particularly preferred embodiment of the present invention, the initiator compound is chosen from trimethylolpropane, pentaerythritol and an ethoxylated pentaerythritol, and the chain-extending molecule is dimethylolpropionic acid.

Some of the terminal hydroxyl functions of the dendritic polymers of polyester type used in the nanocapsules of the present invention can bear substituents derived from at least one chain-terminating agent. The fraction of these terminal hydroxyl functions bearing a chain-terminating unit is generally between 1 and 90 mol %, preferably between 10 and 50 mol %, relative to the total number of terminal hydroxyl functions. The choice of a suitable chain-terminating agent makes it possible to modify as desired the physicochemical properties of the dendritic polyesters used in the compositions of the present invention. The said chain-terminating agent can be chosen from a wide variety of compounds capable of forming covalent bonds with the terminal hydroxyl functions.

These compounds encompass, in particular:

i) saturated or unsaturated, aliphatic or cycloaliphatic monocarboxylic acids (or anhydrides),

ii) saturated or unsaturated fatty acids,

iii) aromatic monocarboxylic acids,

iv) diisocyanate monomers or oligomers or addition products thereof,

v) epihalohydrins,

vi) glycidyl esters of a monocarboxylic acid or of a C₁₋₂₄ fatty acid,

vii) glycidyl ethers of C₁₋₂₄ monovalent alcohols,

viii) addition products derived from a saturated or unsaturated, aliphatic or cycloaliphatic mono-, di- or polycarboxylic acid, or from the corresponding anhydrides,

ix) addition products derived from an aromatic mono-, di- or polycarboxylic acid or from the corresponding anhydrides,

x) epoxides of an unsaturated C₃₋₂₄ monocarboxylic acid or of a corresponding triglyceride,

xi) saturated or unsaturated, aliphatic or cycloaliphatic monofunctional alcohols,

xii) aromatic monofunctional alcohols,

xiii) addition products derived from a saturated or unsaturated, aliphatic or cycloaliphatic mono-, di- or polyfunctional alcohol, and

xiv) addition products derived from an aromatic mono-, di- or polyfunctional alcohol.

Examples of chain-terminating agents which may be mentioned are lauric acid, linseed fatty acids, soybean fatty acids, tallow fatty acids, dehydrogenated castor oil fatty acids, crotonic acid, capric acid, caprylic acid, acrylic acid, methacrylic acid, benzoic acid, para-tert-butylbenzoic acid, abietic acid, sorbinic acid, 1-chloro-2,3-epoxypropane, 1,4-dichloro-2,3-epoxybutane, epoxidized soybean fatty acids, trimethylolpropane diallyl ether maleate, 5-methyl-1,3-dioxane-5-methanol, 5-ethyl-1,3-dioxane-5-methanol, trimethylolpropane diallyl ether, pentaerythrityl triallyl ether, pentaerythrityl triacrylate, triethoxylated pentaerythrityl triacrylate, toluene 2,4-diisocyanate, toluene 2,6-diisocyanate, hexa-methylene diisocyanate or isophorone diisocyanate.

Among these chain-terminating agents which are particularly preferred are capric acid and caprylic acid or a mixture thereof.

The dendritic polymers of polyester type containing terminal hydroxyl functions and optionally bearing chain-terminating groups are known and are sold by the company Perstorp.

Among the polymers which it is particularly preferred to use in the present invention are:

a dendritic polyester obtained by poly-condensation of dimethylolpropionic acid with trimethylolpropane and which is free of chain-terminating agents, for example the product sold under the name “Boltorn® H40 (TMP core)” by the company Perstorp;

a dendritic polyester obtained by poly-condensation of dimethylolpropionic acid with polyoxyethylenated pentaerythritol (on average 5 units of ethylene oxide to each hydroxyl function), which is free of a chain-terminating agent, for example the product sold under the name “Boltorn® H30” by the company Perstorp;

a generation 3 dendritic polyester obtained by polycondensation of dimethylolpropionic acid with polyoxyethylenated pentaerythritol (on average 5 units of ethylene oxide to each hydroxyl function), 50% of the hydroxyl functions of which are esterified with C₈₋₁₀ acids and in particular capric acid and caprylic acid (“Boltorn® H30 (esterified)” sold by the company Perstorp).

Among these three polymers, the last one is the one most particularly preferred.

5) The non-biodegradable polymers that may be used according to the invention may be chosen from any polymer that is not degraded by the enzymes of the skin, and especially those mentioned in document EP-A-557 489. Among the non-biodegradable polymers that may be mentioned in particular are copolymers of vinyl chloride and of vinyl acetate, and copolymers of methacrylic acid and of methyl methacrylate, polyvinyl acetophthalate, cellulose acetophthalate, crosslinked polyvinylpyrrolidone/vinyl acetate copolymers, polyethylenes/vinyl acetates, polyacrylonitriles, polyacrylamides, polyethylene glycols, polyamides, polyethylenes, polypropylenes and polyorganosiloxanes, without this list being limiting.

The nanocapsules that may be formed from the above polymers comprise in their lipid core an oily solvent chosen from the various categories mentioned in claim 1.

Among these solvents, preferred examples are given below:

as 2-alkyl alkanols, the following may preferably be used: butyloctanol, hexyldecanol, octyldecanol, isostearyl alcohol, octyldodecanol, decyltetradecanol, undecylpentadecanol, dodecylhexadecanol, tetradecyloctadecanol, hexyldecyloctadecanol, tetradecyleicosanol, cetylarachidol and the mixture of isocetyl alcohol, isostearyl alcohol and isoarachidyl alcohol. All these compounds are commercially available, from Condea Vista under the trade name Isofol®, from Exxon Chemical under the name Exxal® or from Jarchem under the name Jarcol®. The compound available from Henkel under the trade name Eutanol G may also be used. Esters of the said alcohols that may be mentioned include: octyldodecyl octanoate; hexyldecyl caprylate; hexyldecyl laurate; hexyldecyl palmitate; hexyldecyl stearate; and octyldodecyl meadow foamate, which is an ester of octyldodecanol and of fatty acids derived from Limnanthes Alba germ oil;

esters of fatty acids or of fatty alcohols that may be mentioned include: isopropyl palmitate, isostearyl neopentanoate and octyl palmitate;

an N-acyl amino acid ester of a fatty alcohol that will preferably be used is isopropyl N-lauroyl sarcosinate;

triglycerides and oils containing them that are preferred include octanoic acid triglycerides or sunflower oil, maize oil, soybean oil, marrow oil, grape seed oil, sesame seed oil, hazelnut oil, apricot oil, macadamia oil, arara oil, castor oil, avocado oil, caprylic/capric acid triglycerides such as those sold by the company Stearineries Dubois or those sold under the names Miglyol 810, 812 and 818 by the company Dynamit Nobel, jojoba oil and shea butter oil;

a liquid ether of a fatty alcohol and of polypropylene glycol that is advantageously used is polypropylene glycol stearyl ether containing 15 propylene glycol units; and

a tocopheryl ester that is preferably used is tocopheryl acetate.

However, these examples are not limiting.

The preparation process used for the manufacture of the nanocapsules is preferably that described in one of the patent applications mentioned above, in particular in patent application EP-0 274 961.

One particular process comprises the following steps:

(a) the steroid and the oily solvent in which it will be dissolved are mixed with a water-miscible organic solvent (for example acetone), with the polymer which will form the shell of the nanocapsules and optionally with an amphiphilic lipid capable of forming a liquid crystal phase to form an organic phase;

(b) this organic phase is introduced with stirring into an aqueous phase containing a hydrophilic surfactant. A spontaneous emulsion then forms. The water-insoluble polymer precipitates around the oil globule and the amphiphilic lipid forms a liquid crystal phase surrounding the oil globule encapsulated by the polymer;

(c) the organic solvent and some of the water are evaporated off, so as to concentrate the suspension of nanocapsules. A suspension of nanocapsules containing about 5% by weight of the mixture of oily solvent and of steroid are generally obtained.

The amphiphilic lipid used in step (a) above is a compound capable of spontaneously forming on contact with water a lyotropic liquid crystal phase of lamellar type. The purpose of using it is to facilitate the formation of the capsules, but above all to improve the stability of the nanocapsules and of the encapsulation, via its deposition at the polymer shell/outer aqueous phase interface. Preferably, without this being limiting, mention will be made of phospholipids such as soybean or egg lecithins optionally enriched in phosphatidylcholine and oxyethylenated and/or oxypropylenated silicone surfactants.

This type of silicone surfactant is a silicone compound comprising at least one oxyethylene chain —OCH₂CH₂— and/or oxypropylene chain —OCH₂CH₂CH₂—. As silicone surfactants that may be used according to the present invention, mention may be made of those described in documents U.S. Pat. No. 5,364,633 and U.S. Pat. No. 5,411,744.

Preferably, the silicone surfactant used according to the present invention is a compound of formula (I):

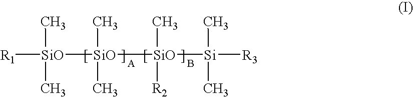

in which:

R₁, R₂ and R₃, independently of each other, represent a C₁-C₆ alkyl radical or a radical

—(CH₂)ₓ—(OCH₂CH₂)_{y}—(OCH₂CH₂CH₂)_{z}—OR₄, at least one radical R₁, R₂ or R₃ not being an alkyl radical; R₄ being a hydrogen, an alkyl radical or an acyl radical;

A is an integer ranging from 0 to 200;

B is an integer ranging from 0 to 50; with the condition that A and B are not simultaneously equal to zero;

x is an integer ranging from 1 to 6;

y is an integer ranging from 1 to 30;

z is an integer ranging from 0 to 5.

According to one preferred embodiment of the invention, in the compound of formula (I), the alkyl radical is a methyl radical, x is an integer ranging from 2 to 6 and y is an integer ranging from 4 to 30.

Examples of silicone surfactants of formula (I) that may be mentioned include the compounds of formula (II):

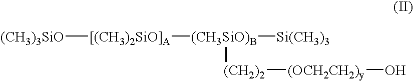

in which A is an integer ranging from 20 to 105, B is an integer ranging from 2 to 10 and y is an integer ranging from 10 to 20.

Examples of silicone surfactants of formula (I) that may also be mentioned include the compounds of formula (III):

HO—(OCH₂CH₂)_{y}—(CH₂)₃—[(CH₃₂SiO]_{A′}—(CH₂)₃—(OCH₂CH₂)_{y}—OH (III)

in which A′ and y are integers ranging from 10 to 20.

Silicone surfactants that may also be used include those sold by the company Dow Corning under the names DC 5329, DC 7439-146, DC 2-5695 and Q4-3667. The compounds DC 5329, DC 7439-146 and DC 2-5695 are compounds of formula (II) in which, respectively, A is 22, B is 2 and y is 12; A is 103, B is 10 and y is 12; A is 27, B is 3 and y is 12.

The compound Q4-3667 is a compound of formula (III) in which A is 15 and y is 13.

Moreover, the outer aqueous phase containing the suspension of nanocapsules may contain, as indicated in step (b) of the above process, a water-soluble hydrophilic surfactant, such as a poloxamer, or a polyol alkyl ester or alkyl ether, so as to facilitate the formation of the nanocapsules. The term “water-soluble surfactant” means that it is soluble to at least 1% in water, the suspension obtained needing to be perfectly clear.

The suspension of nanocapsules according to the invention may then be introduced into a cosmetic or dermatological composition. The invention thus also relates to a cosmetic and/or dermatological composition comprising, in a physiologically acceptable support, a suspension of nanocapsules as defined above.

The fraction represented by the nanocapsules in the cosmetic and/or dermatological compositions of the present invention is generally between 0.1% and 50% by weight and preferably between 0.5% and 25% by weight relative to the total weight of the composition.

The composition according to the invention comprises an effective amount of steroid, which is sufficient to obtain the desired effect, and a physiologically acceptable medium. The expression “physiologically acceptable medium” means a medium that is suitable for topical application to the skin and/or its integuments.

Thus, the concentration of steroid(s) in the composition according to the invention is advantageously between 0.005% and 5% and preferably between 0.05% and 2.5% by weight relative to the total weight of the composition.

The compositions according to the invention may be in any presentation form normally used for topical application to the skin and/or its integuments, for example in the form of an aqueous or aqueous-alcoholic lotion or gel, or a water-in-oil or oil-in-water emulsion or multiple emulsion (for example W/O/W or O/W/O emulsion).

This composition may be more or less fluid and may have the appearance of a white or coloured cream, an ointment, a milk, a lotion, a serum, a paste or a mousse. It may optionally be applied to the skin in aerosol form. It may also be in solid form, for example in the form of a stick. As a variant, it may be in the form of a shampoo or a conditioner.

As examples of oils that may be used in the composition of the invention, mention may be made of:

hydrocarbon-based oils of animal origin, such as perhydrosqualene;

hydrocarbon-based oils of plant origin, such as liquid triglycerides of fatty acids containing from 4 to 10 carbon atoms;

synthetic esters and synthetic ethers, especially of fatty acids, for instance oils of formulae R¹COOR² and R¹OR² in which R¹ represents the fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;

linear or branched hydrocarbons of mineral or synthetic origin, such as volatile or non-volatile liquid paraffins, and derivatives thereof, petroleum jelly, polydecenes, and hydrogenated polyisobutene such as Parleam® oil;

fatty alcohols containing from 8 to 26 carbon atoms, for instance cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleyl alcohol;

partially hydrocarbon-based and/or partially silicone-based fluoro oils, for instance those described in document JP-A-2 295 912;

silicone oils, for instance volatile or non-volatile polymethylsiloxanes (PDMSs) containing a linear or cyclic silicone chain, that are liquid or pasty at room temperature, especially cyclopolydimethylsiloxanes (cyclomethicones) such as cyclohexasiloxane; polydimethylsiloxanes comprising alkyl, alkoxy or phenyl groups, that are pendant or at the end of a silicone chain, these groups containing from 2 to 24 carbon atoms; phenylsilicones, for instance phenyltrimethicones, phenyldimethicones, phenyltrimethylsiloxydiphenylsiloxanes, diphenyldimethicones, diphenylmethyl-diphenyltrisiloxanes, 2-phenylethyltrimethyl-siloxysilicates and polymethylphenylsiloxanes;

mixtures thereof.

In the list of oils mentioned above, the expression “hydrocarbon-based oil” means any oil mainly comprising carbon and hydrogen atoms, and optionally ester, ether, fluoro, carboxylic acid and/or alcohol groups.

The other fatty substances that may be present in the oily phase are, for example, fatty acids containing from 8 to 30 carbon atoms, for instance stearic acid, lauric acid, palmitic acid and oleic acid; waxes, for instance lanolin, beeswax, carnauba wax or candelilla wax, paraffin waxes, lignite wax or microcrystalline waxes, ceresin or ozokerite, synthetic waxes such as polyethylene waxes, Fischer-Tropsch waxes; silicone resins such as trifluoromethyl-C1-4-alkyldimethicone and trifluoropropyldimethicone; and silicone elastomers, for instance the products sold under the name “KSG” by the company Shin-Etsu, under the names “Trefil”, “BY29” or “EPSX” by the company Dow Corning or under the name “Gransil” by the company Grant Industries.

These fatty substances may be chosen in a varied manner by a person skilled in the art so as to prepare a composition having the desired properties, for example in terms of consistency or texture.

According to one particular embodiment of the invention, the composition containing the nanocapsules containing at least one steroid is a water-in-oil (W/O) or oil-in-water (O/W) emulsion. The proportion of oily phase of the emulsion may range from 5% to 80% by weight and preferably from 5% to 50% by weight relative to the total weight of the composition. The oils, emulsifiers and co-emulsifiers used in the composition in emulsion form are chosen from those conventionally used in cosmetics or dermatology. The emulsifier and the co-emulsifier are generally present in the composition in a proportion ranging from 0.3% to 30% by weight and preferably from 0.5% to 20% by weight, relative to the total weight of the composition. The emulsion may also contain lipid vesicles.

The emulsions generally contain at least one emulsifier chosen from amphoteric, anionic, cationic and nonionic emulsifiers, used alone or as a mixture. The emulsifiers are chosen in a suitable manner depending on the emulsion to be obtained (W/O or O/W).

The compositions of the invention may also contain known cosmetic and/or dermatological adjuvants, such as pH regulators, preserving agents, thickeners, colorants, fragrances, fillers, UV-screening agents, other active agents, pigments, odour absorbers and dyestuffs. The amounts of these various adjuvants are those conventionally used in the field under consideration, for example from 0.01% to 20% of the total weight of the composition. Depending on their nature, these adjuvants may be introduced into the fatty phase or into the aqueous phase.

Needless to say, a person skilled in the art will take care to select this or these optional additional compound(s), and the amount thereof, such that the advantageous properties intrinsically associated with the cosmetic or dermatological composition in accordance with the invention are not, or are not substantially, adversely affected by the envisaged addition(s).

As fillers that may be used in the composition of the invention, examples that may be mentioned, besides the pigments, include silica powder; talc; polyamide particles and especially those sold under the name Orgasol by the company Atochem; polyethylene powders; microspheres based on acrylic copolymers, such as those made of ethylene glycol dimethacrylate/lauryl methacrylate copolymer, sold by the company Dow Corning under the name Polytrap; expanded powders such as hollow microspheres and especially the microspheres sold under the name Expancel by the company Kemanord Plast or under the name Micropearl F 80 ED by the company Matsumoto; silicone resin microbeads such as those sold under the name Tospearl by the company Toshiba Silicone; and mixtures thereof. These fillers may be present in amounts ranging from 0 to 20% by weight and preferably from 1% to 10% by weight relative to the total weight of the composition.

The composition according to the invention finds a particular application in caring for the skin and/or its integuments, especially the hair, and/or mucous membranes.

The present invention thus relates also to the cosmetic use of the composition mentioned above for preventing and/or treating the signs of intrinsic or photo-induced ageing of the skin.

The invention also relates to the cosmetic use of this composition for preventing and/or treating baldness or hair loss.

The invention also relates to the use of the composition mentioned above for the manufacture of a preparation for preventing and/or treating atrophy of the skin or of mucous membranes.

The invention will now be illustrated by the non-limiting examples that follow. In these examples, the amounts are indicated as weight percentages, unless otherwise mentioned.

### EXAMPLE 1

### Solubility of DHEA

This example is intended to demonstrate that DHEA is dissolved in the oily solvent contained in the nanocapsules according to the invention at a level higher than its maximum solubility in the non-encapsulated oily solvent.

a) Solubility of DHEA in Various Oily Solvents

The solubility measurement was performed in the following manner. DHEA was dispersed, in excess, in the solvent under consideration, and preheated to 60° C. This temperature was maintained for one hour with stirring using a magnetic bar. The suspension was then cooled to room temperature (RT, 25° C.). After 24 hours, the suspension was centrifuged to remove the undissolved DHEA crystals. The supernatant was taken up and the absence of DHEA crystals was checked by cross-polarized optical microscopy. This supernatant was then analysed by HPLC. It is considered that the amount of DHEA detected corresponds to its maximum solubility in the solvent under consideration.

The results are collated in the table below.

| Chemical name | RT solubility |
|---|---|
| Vitamin E acetate | 5% |
| Octyldodecanol | 4.77% |
| PPG-15 stearyl ether | 2.68% |
| Capric/caprylic triglycerides | 1.77% |
| α-Tocopherol | 1.65% |
| Sesame oil | 1.40% |
| Isopropyl palmitate | 1.37% |
| Isostearyl neopentanoate | 1.30% |
| Jojoba oil | 1.10% |
| Octyl palmitate | 1% |

For comparative purposes, the solubility of DHEA is 0% in liquid paraffin, 0.83% in cetearyl isononanoate, 0.04% in cyclopentasiloxane, 0.17% in polydimethylsiloxane (200-350 cs) and 0.10% in squalane. These lipophilic compounds are therefore not good solvents for DHEA.

b) Solubility of DHEA in Encapsulated Oily Solvents

α) in Vitamin E Acetate

A suspension of nanocapsules having the composition below is prepared:

| | |
|---|---|
| DHEA | 0.5% |
| Vitamin E acetate | 5% |
| Polycaprolactone | 1% |
| Soybean lecithin | 1% |
| Poloxamer 188 | 0.5% |
| Distilled water | 92% |

This suspension was prepared in the following manner:

The DHEA, the vitamin E acetate, the polycaprolactone, and the soybean lecithin are dissolved in 200 ml of acetone. This clear solution is then introduced with stirring into 300 ml of water containing 0.5 g of poloxamer 188. A spontaneous emulsion forms. The acetone and some of the water are evaporated off under reduced pressure. A suspension of nanocapsules 245 nm in size is obtained, which contains a 10% solution of DHEA in vitamin E acetate, whereas the maximum solubility of DHEA in vitamin E acetate does not exceed 5%. The suspension shows no sign of recrystallization of the DHEA at room temperature and at 45° C. over more than 15 days.

β) In Octyldodecanol

A suspension of nanocapsules having the composition below is prepared:

| | |
|---|---|
| DHEA | 0.75% |
| Octyldodecanol | 5% |
| Polycaprolactone | 1% |
| Soybean lecithin | 1% |
| Poloxamer 188 | 0.5% |
| Distilled water | 91.25% |

The above suspension is obtained according to the process described above. The nanocapsules obtained are 176 nm in size and contain a 15% solution of DHEA in octyldodecanol, whereas the maximum solubility of DHEA in octyldodecanol does not exceed 4.7%. The suspension shows no sign of recrystallization of the DHEA at room temperature and at 45° C. over more than 15 days.

### EXAMPLE 2

### Cosmetic Composition

Fatty Phase A:

| | |
|---|---|
| Glyceryl stearate | 2.5% |
| PEG-8 stearate | 2.5% |
| Stearic acid | 1% |
| Preserving agents | 0.1% |
| Myglyol 812 | 10% |
| Apricot oil | 8% |
| Cyclopentasiloxane | 6% |

Aqueous Phase B:

| | |
|---|---|
| Triethanolamine | 0.25% |
| Preserving agents | 0.2% |
| Glycerol | 3% |
| Phenoxyethanol | 0.5% |
| Water | qs 80% |

Phase C:

| | |
|---|---|
| Carbopol 980 | 0.15% |
| Water | 9.6% |
| 10% sodium hydroxide | 0.25% |

Phase D:

| | |
|---|---|
| Suspension of nanocapsules of Example 1 α) | 10% |

The above composition is prepared in the following manner:

Phases A and B are prepared separately and brought to 70° C. Phase B is added to phase A with stirring at 70° C. After 30 minutes, the mixture is homogenized twice. A fluid oil-in-water emulsion in which the size of the oil globules is between 1 and 100 nm is obtained. The temperature is returned to 25° C. and phase C is then added, dispersed using a defloculator. Finally, phase D is added to the emulsion obtained, with gentle stirring.

A white day cream which may be applied once or twice a day as an anti-ageing cream is obtained.

1. An aqueous suspension of nanocapsules comprising water and a plurality of nanocapsules, wherein the nanocapsules comprise a polymer shell and a lipid core comprising an oily solvent, wherein the lipid core comprises at least one steroid selected from the group consisting of DHEA, a chemical precursor of DHEA a biological precursor of DHEA, chemical derivative of DHEA and a metabolic derivative of DHEA, and the oily solvent comprises at least one compound selected from the group consisting of:
a 2-alkyl alkanol containing from 12 to 36 carbon atoms or an ester of the alcohol;
a fatty acid ester in which the acid function contains from 8 to 26 carbon atoms and the alcohol function contains from 2 to 8 carbon atoms;
a fatty alkyl ester in which the acid function contains from 2 to 8 carbon atoms and the alcohol function contains from 8 to 26 carbon atoms;
an ester of an N-acyl amino acid and one or more fatty alcohols;
a triglyceride derived from at least one acid containing from 6 to 20 carbon atoms, a plant oil containing the triglyceride, or both;
a liquid ether of a fatty alcohol and a polypropylene glycol; and
a tocopherol, a tocopheryl ester or both.

2. The suspension according to claim 1, wherein the steroid is DHEA.

3. The suspension according to claim 1, comprising at least one of 7αOH-DHEA, 7βOH-DHEA or 7-keto-DHEA.

4. The suspension according to claim 1, comprising a 3-alkyl ester of 7-oxo-DHEA.

5. The suspension according to claim 4, comprising 3β-acetoxy-7-oxo-DHEA.

6. The suspension according to claim 1, comprising a chemical derivative of DHEA formula (1): 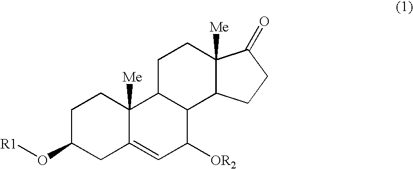
in which:
R₁ and R₂ are independently:
a saturated or unsaturated, linear, branched or cyclic C₁-C₁₂ alkyl group optionally containing one or more hetero atoms, and optionally substituted with one or more of a —OR′, —SR′, —COOR′, —NR′R′, halogen, sulphate, phosphate, aryl, or heterocycle group;
an alkylcarbonyl group, the C₁-C₂₄ alkyl portion of which is saturated or unsaturated, linear, branched or cyclic, and optionally substituted with one or more groups —OR′, —SR′, —COOR′, —NR′R′, halogen, sulphate, phosphate, aryl, or heterocycle group;
an arylcarbonyl group, or an arylalkylcarbonyl group, optionally substituted with one or more of a —OR′, —SR′, —COOR′, —NR′R′, halogen, aryl, or heterocycle group;
a group O═P(OH)OR′;
a group (O)₂SOR′;
a trialkylsilyl group of formula SiR′₃ in which the 3 groups R′ may be identical or different;
a carbonyloxyalkyl group (R′OCO);
a carbonylaminoalkyl group (R′NHCO);
in which R′ a hydrogen atom, a saturated or unsaturated, linear, branched or cyclic C₁-C₁₂ and optionally containing one or more hetero atoms, optionally functionalized with one or more of a —OR″, —COOR″, halogen, —NR″R″; or an aryl group, optionally functionalized with one or more of a —OR″, —COOR″, halogen or —NR″R″ group;
R″ is a hydrogen atom or a saturated or unsaturated, linear, branched or cyclic alkyl chain,
wherein each of the groups —NR′R′ and —NR″R″, the substituents R′ or R″, respectively, are identical or different.

7. The suspension according to claim 6, wherein the chemical derivative of DHEA is 3-O-acetyl-7-benzoyloxy-dehydroepiandrosterone.

8. The suspension according to claim 1, comprising at least one of a sapogenin, a plant extract containing a sapogenin or a chemical derivative of a sapogenin.

9. The suspension according to claim 8, comprising diosgenin.

10. The suspension according to claim 8, comprising hecogenin acetate.

11. The suspension according to claim 8, comprising an extract of wild yam root.

12. The suspension according to claim 1, wherein the content of the steroid in the nanocapsules is higher than the maximum solubility level of the steroid in the oily solvent.

13. The suspension according to claim 1, wherein the polymer shell comprises at least one polymer selected from the group consisting of poly-L- and DL-lactides and polycaprolactones, polyglycolides and copolymers thereof, polymers derived from the polymerization of alkyl cyanoacrylate wherein the alkyl chain contains from 2 to 6 carbon atoms; synthetic or natural water-dispersible anionic polymers; polyesters of the poly(alkylene adipate) type; dendritic polymers; copolymers of vinyl chloride and of vinyl acetate; copolymers of methacrylic acid and of methyl methacrylate, polyvinyl acetophthalate, cellulose acetophthalate, crosslinked polyvinylpyrrolidone/vinyl acetate copolymers, polyethylene-vinyl acetates, polyacrylonitriles, polyacrylamides, polyethylene glycols, polyamides, polyethylenes, polypropylenes and polyorganosiloxanes.

14. The suspension according to claim 13, comprising a synthetic water-dispersible anionic polymer selected from the group consisting of polyesters, poly(esteramides), polyurethanes and vinyl copolymers, all bearing at least one of a carboxylic acid or sulphonic acid functions, and natural water-dispersible anionic polymers selected from the group consisting of shellac resin, sandarac gum and dammar resins.

15. The suspension according to claim 14, comprising one or more synthetic water-dispersible anionic polymers selected from the group consisting of copolyesters comprising at least units derived from isophthalic acid, sulphoaryldicarboxylic acid and diethylene glycol.

16. The suspension according to claim 13, comprising a poly(ethylene adipate) with a weight-average molar mass of about 10 000.

17. The suspension according to claim 13, comprising a 3^{rd} generation dendritic polyester obtained by polycondensation of dimethylolpropionic acid with polyoxyethylenated pentaerythritol having on average 5 ethylene oxide units on each hydroxyl function, 50% of the hydroxyl functions of which are esterified with C₈₋₁₀ acids and especially capric and caprylic acids.

18. The suspension according to claim 1, comprising a 2-alkyl alkanol selected from the group consisting of butyloctanol, hexyldecanol, octyldecanol, isostearyl alcohol, octyldodecanol, decyltetradecanol, undecylpentadecanol, dodecylhexadecanol, tetradecyloctadecanol, hexyldecyloctadecanol, tetradecyleicosanol, cetylarachidol and a mixture of isocetyl alcohol, isostearyl alcohol and isoarachidyl alcohol.

19. The suspension according to claim 1, comprising one or more esters of fatty acid or a fatty alcohol selected from the group consisting of isopropyl palmitate, isostearyl neopentanoate and octyl palmitate.

20. The suspension according to claim 1, comprising isopropyl N-lauroylsarcosinate.

21. The suspension according to any one of claim 1, comprising one or more triglycerides formed from at least one acid containing from 6 to 20 carbon atoms, or a plant oil containing the triglyceride, selected from the group consisting of caprylic/capric triglycerides, sesame oil and jojoba oil.

22. The suspension according to claim 1, comprising a polypropylene glycol stearyl ether containing 15 propylene glycol units.

23. The suspension according to claim 1, comprising tocopheryl acetate.

24. The suspension according to claim 1, wherein the size of the nanocapsules is between 10 nm and 1000 nm.

25. The suspension according to claim 24, wherein the size of the nanocapsules is between 30 nm and 500 nm.

26. A cosmetic or dermatological composition comprising, in a physiologically acceptable support, the aqueous suspension of nanocapsules according to claim 1.

27. The cosmetic or dermatological composition according to claim 26, comprising from 0.1% to 50% by weight of the nanocapsules.

28. The cosmetic or dermatological composition according to claim 27, comprising from 0.5% to 25% by weight of the nanocapsules.

29. The cosmetic or dermatological composition according to claim 1, comprising from 0.005% to 5% by weight of the steroid relative to the total weight of the composition.

30. The cosmetic or dermatological composition according to claim 29, comprising from 0.05% to 2.5% by weight of the steroid relative to the total weight of the composition.

31. The cosmetic or dermatological composition according to claim 26, in the form of a water-in-oil or oil-in-water emulsion.

32-34. (Canceled).

35. A process for manufacturing the suspension of nanocapsules according to claim 1, comprising:
(a) mixing the steroid and the oily solvent with a water-miscible organic solvent, with the polymer which will form the shell of the nanocapsules and optionally with an amphiphilic lipid capable of forming a liquid crystal phase to form an organic phase;
(b) introducing the organic phase with stirring into an aqueous phase containing a hydrophilic surfactant;
(c) evaporating the organic solvent and a portion of the water.

36. The process according to claim 35, wherein the amphiphilic lipid is a phospholipid optionally enriched in at least one of a phosphatidylcholine, an oxyethylenated surfactant and a oxypropylenated silicone surfactant.

37. The process according to claim 36, wherein the silicone surfactant is of formula (II): 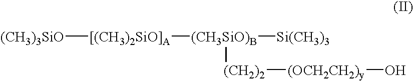
in which A is an integer ranging from 20 to 105, B is an integer ranging from 2 to 10 and y is an integer ranging from 10 to 20,
or a compound of formula (III):
HO—(OCH₂CH₂)_{y}—(CH₂)₃[(CH₃)₂SiO]_{A′}—(CH₂)₃—(OCH₂CH₂)_{y}—OH (III)
in which A′ and y are integers ranging from 10 to 20.

38. The suspension according to claim 6, wherein at least one of R₁ or R₂ is a group substituted with a heterocycle selected from the group consisting of indole, a pyrimidine, a piperidine, a morpholine, a pyran, a furan, a piperazine and a pyridine.

39. The composition of claim 6, wherein at least one of R₁ or R₂ is a phenylcarbonyl group.

40. The composition of claim 6, wherein at least one of R₁ or R₂ is a benzylcarbonyl group.

41. The composition of claim 6, wherein at least one of R₁ or R₂ is substituted with a C₁-C₆ alkyl group.

42. The composition of claim 6, wherein at least one of R₁ or R₂ is substituted with a R′ group functionalized with a phenyl group.

43. The composition of claim 6, wherein at least one of R₁ or R₂ is substituted with a R′ group functionalized with a C₁-C₆-containing R″ group.

44. The process according to claim 36, wherein the phospholipid is a soybean lecithin or an egg lecithin.

45. The process according to claim 35, wherein the organic solvent is acetone.
